(19) United States
(12) Patent Application Publication
(10) Pub. No.: US 2024/0156385 A1
(43) Pub. Date: May 16, 2024

(54) Neurotrophic Electrode Array

(71) Applicants: Neural Logistics LLC, Duluth, GA (US); NeuroNexus Technologies, Inc., Ann Arbor, MI (US)

(72) Inventors: Philip R. Kennedy, Duluth, GA (US); Jamille Hetke, Ann Arbor, MI (US)

(21) Appl. No.: 18/384,628

(22) Filed: Oct. 27, 2023

Related U.S. Application Data
(60) Provisional application No. 63/419,941, filed on Oct. 27, 2022.

(51) Int. Cl. A61B 5/268; H05K 1/02; H05K 1/03

(52) U.S. Cl. CPC A61B 5/268; H05K 1/0296; H05K 1/0346; A61B 2562/125; H05K 2201/0154; H05K 2201/09027

(57) ABSTRACT

An electrode array includes a fan-shaped substrate member. The fan-shaped substrate member includes a dielectric material and that has a triangular portion with a convexly curved base from which a first side and an opposite second side extend to a truncated apex that includes a concavely curved surface. An elongated lead member includes the dielectric material and extends from the base adjacent to a selected one of the first side and the second side. The elongated lead member is contiguous with the fan-shaped substrate member. Each of a plurality of wires is embedded in the fan-shaped substrate member and the elongated lead member. Each of a corresponding plurality of electrodes is electrically coupled to a different one of the plurality of wires. Each of the corresponding plurality of electrodes includes an exposed surface.

### BACKGROUND OF THE INVENTION

### 1. Field of the Invention

The present invention relates to neural electrodes and, more specifically, to a 16-channel neural electrode array disposed on a fan-shaped substrate.

### 2. Description of the Related Art

The traditional neurotrophic electrode has several advantages over metal electrodes, including: stable single units with no need to reclassify them over time, long term survival (e.g., at least 13 years) with no scarring and no loss of signal and it is similar to rat and monkey histological analyses. However, it has an inadequate number of single units—about 20 per electrode wire pair.

### SUMMARY OF THE INVENTION

The disadvantages of the prior art are overcome by the present invention which, in one aspect, is an electrode array that includes a fan-shaped substrate member. The fan-shaped substrate member includes a dielectric material and that has a triangular portion with a convexly curved base from which a first side and an opposite second side extend to a truncated apex that includes a concavely curved surface. An elongated lead member includes the dielectric material and extends from the base adjacent to a selected one of the first side and the second side. The elongated lead member is contiguous with the fan-shaped substrate member. Each of a plurality of wires is embedded in the fan-shaped substrate member and the elongated lead member. Each of a corresponding plurality of electrodes is electrically coupled to a different one of the plurality of wires. Each of the corresponding plurality of electrodes includes an exposed surface.

In another aspect, the invention is an electrode unit that includes a fan-shaped substrate member. The fan-shaped substrate member includes a dielectric material and that has a triangular portion with a convexly curved base from which a first side and an opposite second side extend to a truncated apex that includes a concavely curved surface. An elongated lead member includes the dielectric material and extends from the base adjacent to a selected one of the first side and the second side. The elongated lead member is contiguous with the fan-shaped substrate member. Each of a plurality of wires is embedded in the fan-shaped substrate member and the elongated lead member. Each of a corresponding plurality of electrodes is electrically coupled to a different one of the plurality of wires. Each of the corresponding plurality of electrodes includes an exposed surface. A glass cone defines a cavity therein that opens to an open base and an opposite open vertex. The fan-shaped substrate member is rolled into a cone shape and is disposed in the cavity so that the elongated lead member extends out of the open base.

In yet another aspect, the invention is a method of making an electrode unit, in which a plurality of electrically conductive electrodes is printed onto a first flexible dielectric layer so that the electrically conductive electrodes are distributed in first fan shape. A plurality of electrically conductive wires is printed onto the first flexible dielectric layer wherein each of the electrically conductive wires is electrically coupled to a different one of the electrically conductive electrodes. A second flexible dielectric layer is applied onto the first flexible dielectric layer so as to cover each of the plurality of wires and so as to expose a portion of each of the plurality of electrically conductive electrodes. The first flexible dielectric layer and the second flexible dielectric layer are formed into a second fan shape that corresponds to the first fan shape, thereby making a fan shaped electrode array. The fan-shaped electrode array is rolled into a cone shape, thereby making a cone-shaped electrode array. The cone-shaped electrode array is placed into a glass cone that defines a cavity therein and that opens to an open base and an opposite open vertex. A portion of each of the plurality of electrically conductive wires extends out of the cavity through the open base of the glass cone.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a schematic diagram of one embodiment of an electrode array on a fan-shaped substrate in an unrolled state.

FIG. 1B is a cross sectional diagram of the embodiment shown in FIG. 1A, taken along line 1B-1B.

FIG. 2 is a schematic diagram of one embodiment of an electrode array on a fan-shaped substrate in a rolled state.

FIG. 3 is a schematic diagram of one embodiment of an electrode array unit on a fan-shaped substrate in a rolled state and disposed in a cone.

FIGS. 4A-4D are side elevational view schematic diagrams demonstrating one method of making an electrode array.

FIG. 5 is a photograph of one experimental embodiment of an electrode array on a fan-shaped substrate in a rolled state and disposed in a cone.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

U.S. Pat. No. 10,575,750 is incorporated herein by reference for the purpose of disclosing serpentine undulated ribbons and methods of making neurotrophic electrodes.

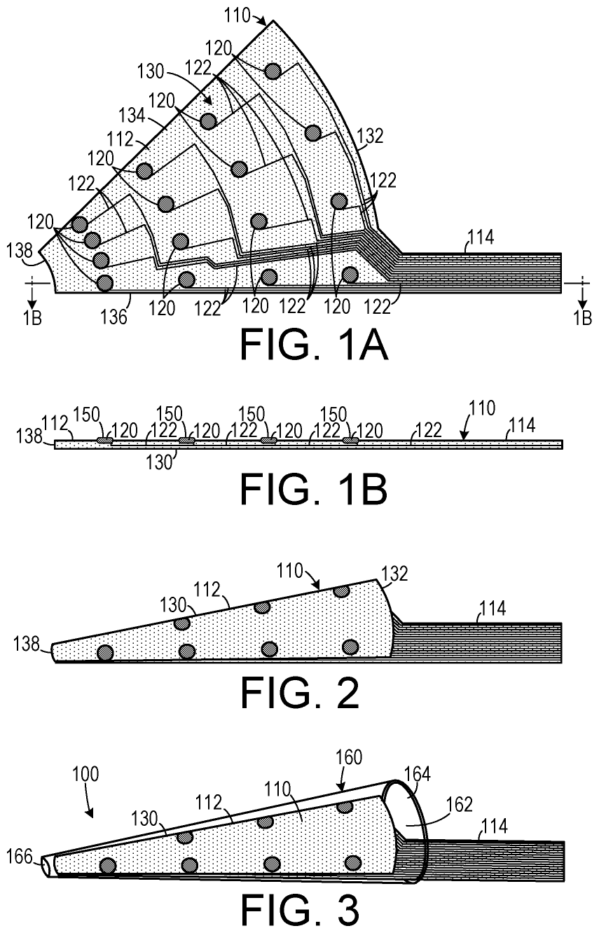

As shown in FIGS. 1A and 1B, one embodiment of an electrode array 110 includes a fan-shaped substrate member 112 with an elongated lead member 114 extending therefrom. The fan-shaped substrate member 112 has a triangular portion 130 with a convexly curved base 132 from which a first side leg 134 and an opposite second side leg 136 extend to a truncated apex 138 that includes a concavely curved surface. The elongated lead member 114 extends from the base 132 and is contiguous with the fan-shaped substrate member 112.

The fan-shaped substrate member 112 and the lead member 114 can include a flexible dielectric biocompatible polymer, such as polyimide. An array of electrodes 120, for sensing neural impulses in the brain of an implanted subject, is disposed across the fan-shaped substrate member 112 and is exposed to an outer surface thereof. Each of a plurality of wires 122 is electrically coupled to a different one of the electrodes 120 and each extends through the lead member 114. Each of the electrodes 120 has an exposed surface 150 that can be in contact with neurites or axons after implantation. The wires 122 are each insulated by the fan-shaped substrate member 112 and the lead member 114. The wires 122 may be coupled to leads from neural potential sensing devices (not shown).

As shown in FIG. 2, the fan-shaped substrate member 112 can be rolled into a cone shaped structure 130 and, as shown in FIG. 3, the resulting cone shaped electrode array 110 can be placed in an insulating cone 160 (such as a glass cone) to form an implantable electrode array unit 100. The insulating cone 160 defines a cavity 162 that is generally complementary in shape to the cone shaped structure 130 and that opens to an open base 164 and an opposite open vertex 166. The cone shaped structure 130 is disposed in the cavity 162 with the lead member 114 extending out of the open base 164. A trophic factor can be placed around the electrodes 120 in the cavity 162 so as to encourage neurite growth once the electrode array unit 100 has been implanted in the neural tissue of the subject.

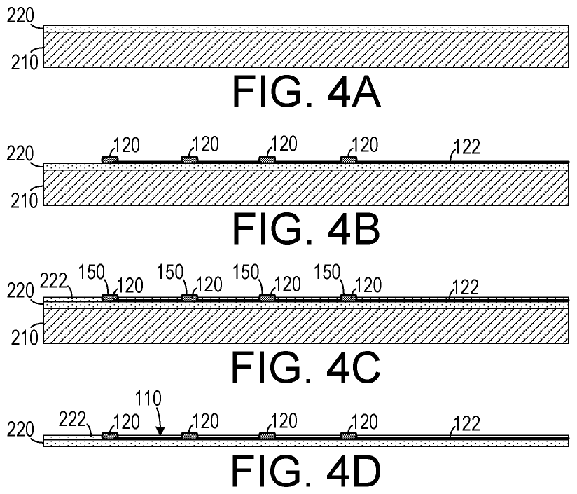

As shown in FIGS. 4A-4D, in one representative method of making the electrode array 110, a first layer of flexible dielectric biocompatible polymer 220, such as polyimide, is applied to a rigid plate 210 (such as a glass plate). This can be done, e.g., by spin coating or any other method of generating polymer films. The wires 122 and electrodes 120 are printed onto the first layer of flexible dielectric biocompatible polymer 220. A second layer of flexible dielectric biocompatible polymer 222 is applied to the first layer of flexible dielectric biocompatible polymer 220 so as to insulate the wires 122 while leaving a surface 150 of the electrodes 120 exposed. If the second layer of flexible dielectric biocompatible polymer 222 covers all of the electrodes 120, then enough of the polymer to expose the electrodes 120 can be removed, e.g., by etching. If necessary, the first layer of flexible dielectric biocompatible polymer 220 and the second layer of flexible dielectric biocompatible polymer 222 are cured and then the polymer layers can be cut into a fan shape. The resulting electrode array 110 is then removed from the rigid plate 210.

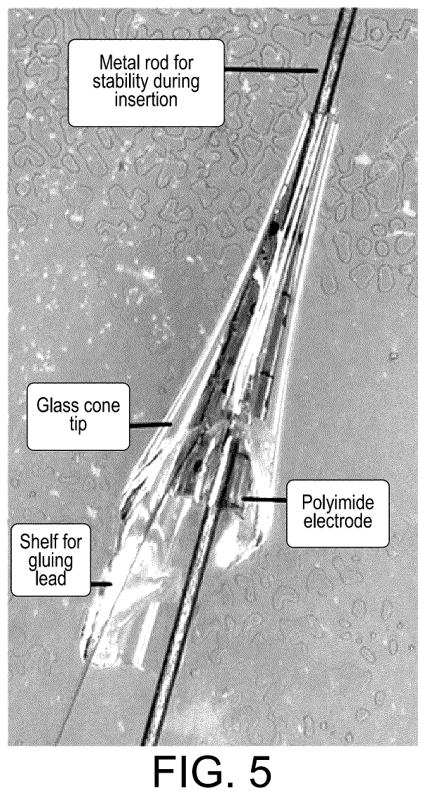

An experimental embodiment is shown in FIG. 5, in which a metal rod may be passed through the cone to stabilize the implantable electrode array unit during insertion into the glass cone. This embodiment includes an electrode with 16 contacts inside the glass tip. This has been achieved by using a polyimide substrate that is rolled up before being placed inside the glass cone that is 2 mm in length×0.5 mm (upper end) and 0.05 mm (deep end). The polyimide lead can be serpentine allowing 3D movement that reduces the strain on the implant and assists with longevity. Trophic factors were placed inside the glass cone prior to implantation in rat vibrissa cortex. It is expected that the electrode will provide several hundred clearly identified single units per electrode.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. An electrode array, comprising:
(a) a fan-shaped substrate member that includes a dielectric material and that has a triangular portion with a convexly curved base from which a first side and an opposite second side extend to a truncated apex that includes a concavely curved surface
(b) an elongated lead member that includes the dielectric material and that extends from the base adjacent to a selected one of the first side and the second side, is contiguous with the fan-shaped substrate member;
(c) a plurality of wires that are each embedded in the fan-shaped substrate member and the elongated lead member; and
(d) a corresponding plurality of electrodes, each of which is electrically coupled to a different one of the plurality of wires and each of which includes an exposed surface.

2. The electrode array of claim 1, wherein the dielectric material comprises polyimide.

3. The electrode array of claim 1, wherein the fan-shaped substrate member is rolled into a cone shape and further comprising an insulating cone, the insulating cone defining a cavity therein that opens to an open base and an opposite open vertex, the fan-shaped substrate member disposed in the cavity so that the elongated lead member extends out of the open base.

4. The electrode array of claim 3, wherein the insulating cone comprises a glass.

5. The electrode array of claim 3, further comprising a trophic factor placed inside of the cavity defined by the insulating cone.

6. An electrode unit, comprising:
(a) a fan-shaped substrate member that includes a dielectric material and that has a triangular portion with a convexly curved base from which a first side and an opposite second side extend to a truncated apex that includes a concavely curved surface
(b) an elongated lead member that includes the dielectric material and that extends from the base adjacent to a selected one of the first side and the second side, is contiguous with the fan-shaped substrate member;
(c) a plurality of wires that are each embedded in the fan-shaped substrate member and the elongated lead member;
(d) a corresponding plurality of electrodes, each of which is electrically coupled to a different one of the plurality of wires and each of which includes an exposed surface; and
(e) a glass cone defining a cavity therein that opens to an open base and an opposite open vertex,
wherein the fan-shaped substrate member is rolled into a cone shape that is disposed in the cavity so that the elongated lead member extends out of the open base.

7. The electrode unit of claim 6, wherein the dielectric material comprises polyimide.

8. The electrode unit of claim 6, further comprising a trophic factor placed inside of the cavity defined by the glass cone.

9. A method of making an electrode unit, comprising the steps of:
(a) printing a plurality of electrically conductive electrodes onto a first flexible dielectric layer so that the electrically conductive electrodes are distributed in first fan shape;
(b) printing a plurality of electrically conductive wires onto the first flexible dielectric layer wherein each of the electrically conductive wires is electrically coupled to a different one of the electrically conductive electrodes;
(c) applying a second flexible dielectric layer onto the first flexible dielectric layer so as to cover each of the plurality of wires and so as to expose a portion of each of the plurality of electrically conductive electrodes;
(d) forming the first flexible dielectric layer and the second flexible dielectric layer into a second fan shape that corresponds to the first fan shape, thereby making a fan shaped electrode array;
(e) rolling the fan-shaped electrode array into a cone shape, thereby making a cone-shaped electrode array; and
(f) placing the cone-shaped electrode array into a glass cone that defines a cavity therein that opens to an open base and an opposite open vertex, wherein a portion of each of the plurality of electrically conductive wires extends out of the cavity through the open base of the glass cone.

10. The method of claim 9, further comprising the step of placing a trophic factor in the cavity defined by the glass cone.

11. The method of claim 9, wherein the step of applying a second flexible dielectric layer comprises spin coating.
